(19) United States
(12) Patent Application Publication
(10) Pub. No.: US 2025/0143641 A1
(43) Pub. Date: May 8, 2025

(54) VITALS MONITORING HANDCUFF APPARATUS

(71) Applicant: Zachary Owen Rebro, Lynden, WA (US)

(72) Inventor: Zachary Owen Rebro, Lynden, WA (US)

(21) Appl. No.: 19/016,619

(22) Filed: Jan. 10, 2025

Related U.S. Application Data
(63) Continuation-in-part of application No. 17/378,657, filed on Jul. 17, 2021, now Pat. No. 12,183,178.
(63) Continuation-in-part of application No. 17/520,250, filed on Nov. 5, 2021.

(51) Int. Cl. A61B 5/00; A61B 5/0205; E05B 75/00; G16H 10/60; G16H 40/67

(52) U.S. Cl. CPC A61B 5/6824; A61B 5/0002; A61B 5/0205; A61B 5/746; E05B 75/00; G16H 10/60; G16H 40/67; A61B 2560/0443

(57) ABSTRACT

A vitals monitoring apparatus coupled or affixed to a set of handcuffs placed on a detained wearer operably connected with a specialized external device and/or companion application to record data comprising vitals measurements, assign said data to a log file, identify trends of vitals measurements, and alert law enforcement officials of an emerging or present medical issue.

### CROSS REFERENCE TO RELATED APPLICATIONS

This Application claims the benefit of and is a continuation-in-part of U.S. Pat. No. 12,183,178 filed Jul. 17, 2021 and titled “VITALS MONITORING HANDCUFF APPARATUS,” and U.S. patent application Ser. No. 17/520,250 filed Nov. 11, 2021 and titled “VITALS MONITORING HANDCUFF APPARATUS AND ASSOCIATED METHOD OF USE,” the contents of which are incorporated by reference in their entirety.

### FIELD OF INVENTION

The present invention relates to an improved set of handcuff devices having a number of features which facilitate the collection and presentation of biometric data of a wearer. These features may include, for example, measuring the heartrate of the wearer, measuring the surface temperature of the wearer's skin, measuring the blood pressure of the wearer, measuring the respiration rate of the wearer, providing the location data of the wearer, those features being controlled and analyzed by a controller unit, and the handcuff device having a communication unit that displays or alerts a law enforcement official to a possible medical situation of the wearer.

### BACKGROUND OF THE INVENTION

Physical restraints, such as handcuffs or other bindings, are often used by law enforcement officials to restrain a detained individual. Often, these devices cover areas on an individual's body that are suited to measuring vitals readings, such as handcuffs covering a wearer's wrists. Currently, prior to transporting a restrained individual, law enforcement officials are trained to manually take vital readings and/or wait for emergency medical service providers to arrive for assessment when a restrained individual shows signs of a medical issue. Additionally, even with proper training, it may be difficult for a law enforcement official to assess and constantly monitor the physical condition of a restrained individual. This is especially true when the law enforcement official is transporting a restrained individual who did not present signs of a medical issue prior to the law enforcement official placing the restrained individual in the vehicle. Thus, there is a long-felt need for a device which can simultaneously restrain an individual, monitor that individual's vital signs, and provide data and/or alerts to the law enforcement official for emerging or present medical issues.

### SUMMARY OF THE INVENTION

The following presents a simplified summary of the present invention to provide a basic understanding of the invention's concepts. This summary is not an extensive overview, and it is not intended to identify critical elements or to limit the scope of this discloser. The sole purpose of this summary is to present some general concepts in a simplified form as a prelude to the detailed description of the invention.

The subject matter disclosed and claimed herein, in one embodiment of the present invention, comprises a vitals monitoring apparatus coupled with a set of handcuffs comprising a housing structure having an exterior and interior, adapted to be coupled and uncoupled with the set of handcuffs used by a law enforcement official for detainment of a wearer, the interior of the housing structure containing a plurality of modules and units, the exterior of the housing structure constructed to generally have a complimentary shape to the set of handcuffs, the exterior of the housing structure configured to contact the wearer's wrist while the set of handcuffs is engaged in a locked position on the wearer's wrist during detainment, and the housing structure being constructed to allow access to the set of handcuff's locking mechanism; at least two sensor modules having an interior portion of each sensor module located within the interior of the housing structure and an exterior portion of each sensor module protruding through the exterior of the housing structure, the exterior portion of each sensor module making contact with the wearer's wrist while the set of handcuffs are engaged in the locked position on the wearer, and each sensor module comprising a sensor adapted to measure at least one vitals measurement and continuously monitor that vitals measurement, wherein each sensor is selected from the group consisting of a SpO2 sensor, photoplethysmography sensor, a bioimpedance sensor, a dermal temperature sensor, an electrocardiogram sensor, and an electrodermal activity sensor, and wherein at least two different types of sensors from the group are selected to be used; a controller unit that is located within the interior of the housing structure, the controller unit being configured to relay instructional programs to the modules and units which cause the modules and units to operate, the controller unit receiving a vitals measurement from each sensor module and, upon detection of at least one vitals measurement outside of given parameters, producing at least one corresponding output signal related to the wearer's at least one vitals measurement outside of given parameters; a communication unit that is located within the interior of the housing structure and receives the at least one corresponding output signal from the controller unit and is adapted to relay the at least one corresponding output signal to the law enforcement official to alert the law enforcement official of the wearer's medical issue via a speaker; and a rechargeable power unit located within the interior of the housing structure that is accessed by a port on the exterior of the housing structure, provides power to the vitals monitoring apparatus's other modules and units, and having a receiver which connects with an exterior power source via the port.

The subject matter disclosed and claimed herein, in another embodiment of the present invention, comprises a system including a vitals monitoring apparatus coupled to a set of handcuffs and a specialized external device, the system comprising a vitals monitoring apparatus coupled with a set of handcuffs comprising a housing structure having an exterior and interior, adapted to be coupled and uncoupled with the set of handcuffs used by a law enforcement official for detainment of a wearer, the interior of the housing structure containing a plurality of modules and units, the exterior of the housing structure constructed to generally have a complimentary shape to the set of handcuffs, the exterior of the housing structure configured to contact the wearer's wrist while the set of handcuffs is engaged in a locked position on the wearer's wrist during detainment, and the housing structure being constructed to allow access to the set of handcuff's locking mechanism; at least two sensor modules having an interior portion of each sensor module located within the interior of the housing structure and an exterior portion of each sensor module protruding through the exterior of the housing structure, the exterior portion of each sensor module making contact with the wearer's wrist while the set of handcuffs are engaged in the locked position on the wearer, and each sensor module comprising a sensor adapted to measure at least one vitals measurement and continuously monitor that vitals measurement, wherein each sensor is selected from the group consisting of a SpO2 sensor, photoplethysmography sensor, a bioimpedance sensor, a dermal temperature sensor, an electrocardiogram sensor, and an electrodermal activity sensor, and wherein at least two different types of sensors from the group are selected to be used; a controller unit that is located within the interior of the housing structure, the controller unit being configured to relay instructional programs to the modules and units which cause the modules and units to operate, the controller unit receiving a vitals measurement from each sensor module and, upon detection of at least one vitals measurement outside of given parameters, producing at least one corresponding output signal related to the wearer's at least one vitals measurement outside of given parameters; a communication unit that is located within the interior of the housing structure and receives the at least one corresponding output signal from the controller unit and is adapted to relay the at least one corresponding output signal to the law enforcement official to alert the law enforcement official of the wearer's medical issue via a speaker; and a rechargeable power unit located within the interior of the housing structure that is accessed by a port on the exterior of the housing structure, provides power to the vitals monitoring apparatus's other modules and units, and having a receiver which connects with an exterior power source via the port; a specialized external device comprising a device attachment structure for attaching to at least one of the law enforcement official's belt, uniform, gear, and vehicle; vitals monitoring apparatus connection method; a speaker; an apparatus holster structure for holstering with the vitals monitoring apparatus; an apparatus charger for charging the vitals monitoring apparatus while holstered; associated processors, memory, circuitry, and a power unit; wherein the communication unit of the vitals monitoring apparatus transmits the wearer's at least one vitals measurement to the specialized external device; wherein, upon detection of a vitals measurement outside of given parameters, the specialized external device sends an alert to the law enforcement official regarding the wearer's medical issue; and wherein the vitals monitoring apparatus is adapted to charge while the vitals monitoring apparatus is holstered in apparatus holster structure of the specialized external device and the vitals monitoring apparatus is engaged with the apparatus charger.

The subject matter disclosed and claimed herein, in another embodiment of the present invention, comprises a system including a vitals monitoring apparatus and a companion application, the system comprising a vitals monitoring apparatus coupled with a set of handcuffs comprising a housing structure having an exterior and interior, adapted to be coupled and uncoupled with the set of handcuffs used by a law enforcement official for detainment of a wearer, the interior of the housing structure containing a plurality of modules and units, the exterior of the housing structure constructed to generally have a complimentary shape to the set of handcuffs, the exterior of the housing structure configured to contact the wearer's wrist while the set of handcuffs is engaged in a locked position on the wearer's wrist during detainment, and the housing structure being constructed to allow access to the set of handcuff's locking mechanism; at least two sensor modules having an interior portion of each sensor module located within the interior of the housing structure and an exterior portion of each sensor module protruding through the exterior of the housing structure, the exterior portion of each sensor module making contact with the wearer's wrist while the set of handcuffs are engaged in the locked position on the wearer, and each sensor module comprising a sensor adapted to measure at least one vitals measurement and continuously monitor that vitals measurement, wherein each sensor is selected from the group consisting of a SpO2 sensor, photoplethysmography sensor, a bioimpedance sensor, a dermal temperature sensor, an electrocardiogram sensor, and an electrodermal activity sensor, and wherein at least two different types of sensors from the group are selected to be used; a controller unit that is located within the interior of the housing structure, the controller unit being configured to relay instructional programs to the modules and units which cause the modules and units to operate, the controller unit receiving a vitals measurement from each sensor module and, upon detection of at least one vitals measurement outside of given parameters, producing at least one corresponding output signal related to the wearer's at least one vitals measurement outside of given parameters; a communication unit that is located within the interior of the housing structure and receives the at least one corresponding output signal from the controller unit and is adapted to relay the at least one corresponding output signal to the law enforcement official to alert the law enforcement official of the wearer's medical issue via a speaker; and a rechargeable power unit located within the interior of the housing structure that is accessed by a port on the exterior of the housing structure, provides power to the vitals monitoring apparatus's other modules and units, and having a receiver which connects with an exterior power source via the port; a companion application installed on at least one of the law enforcement official's personal device or a piece of law enforcement equipment; wherein the companion application allows the law enforcement official to perform at least one of the following actions: review the wearer's current vitals measurements, review trending vitals measurements, review maximum recorded vitals measurements, display the status of the vitals monitoring apparatus, review a log file created once the vitals monitoring apparatus was activated and the handcuffs engaged in the locked position on the wearer for detainment, add notes to the log file, and transmit data to a centralized server.

To the accomplishment of the foregoing and related ends, certain illustrative aspects of the disclosed innovation are described herein in connection with the following description and the annexed drawings. These aspects are indicative of only a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The embodiments of the present invention disclosed herein are illustrated by way of example, and not by way of limitation, in the figures of the accompanying drawings, in which like reference numerals may refer to similar elements.

FIG. 1 illustrates the vitals monitoring handcuff apparatus of the present invention in accordance with the disclosed architecture.

FIG. 2 illustrates another angle of the vitals monitoring handcuff apparatus of FIG. 1.

FIG. 3 illustrates the vitals monitoring handcuff apparatus of FIG. 1 coupled with a set of exemplary handcuffs.

FIG. 4 shows a representational diagram of the circuitry of the vitals monitoring handcuff apparatus depicting the connection of the controller unit to the other modules and units, and the power unit also connected to the other modules and units.

FIG. 5 illustrates the vitals monitoring handcuff apparatus of the present invention comprising two housing structures, each affixed to a cuff of a set of handcuffs.

FIG. 6 details an exemplary method for using a vitals monitoring handcuff apparatus.

### DETAILED DESCRIPTION OF THE PRESENT INVENTION

The innovation is now described with reference to the drawings, wherein reference numerals are used to refer to elements throughout. In the following description, for purposes of explanation, numerous specific details are set forth to provide a thorough understanding of the present invention. It may be evident that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a restraining apparatus that can monitor the vitals of a wearer. Moreover, there is a long-felt need for a restraining device that monitors the wearer's vital signs and alerts law enforcement officials (or other users of the present invention) of emerging or present medical issues. Similar vitals monitoring technologies are used in the medical devices and consumer products fields; however, these devices do not offer physical restraint nor are they adapted to alert law enforcement officials of medical issues.

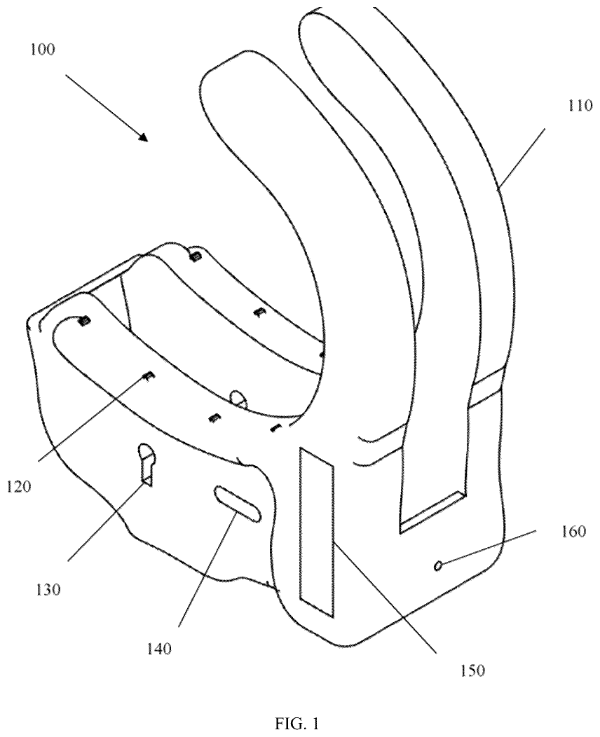

Referring to FIG. 1. In some embodiments, the present invention is a novel vitals monitoring handcuff apparatus 100 that comprises a housing structure 110 that partially encompasses and is coupled to a suitable set of handcuffs. The coupling of the housing structure 110 and the set of handcuffs is configured in a way to allow the set of handcuffs' normal open and close motions to enable a law enforcement official's safe detainment of a wearer of the vitals monitoring handcuff apparatus 100. (In criminal law and as used by law enforcement officials, detainment means to hold an individual in custody for a temporary (meaning “short”) period of time.) For example, the housing structure 110 will couple with a standard set of handcuffs (such as Armament Systems & Procedures (“ASP”) handcuffs). That standard set of handcuffs retains the ability to rotate its rachet system while coupled with the vitals monitoring handcuff apparatus 100. The housing structure 110 is configured in a way that allows it to make contact with the wearer's wrist while the set of handcuffs is in a locked position. The housing structure 110 is a shell that has an exterior and an interior. The interior of the housing structure 110 may be accessed by disassembling the housing structure 110, through access panels, or via the other various holes and ports in the housing structure 110. The set of handcuffs may be of a common variety having a section with a double strand (or check plates) and a section with a single strand (or bow/ratchet), connected by a single strand rivet or fastener.

The housing structure 110 is adapted to have a keyhole access point 130 and/or secondary access point 140 to a locking mechanism in the set of handcuffs. Additionally, the housing structure 110 is adapted to allow a connection portion of the set of handcuffs to connect, through a gap in the housing structure 110, from one cuff portion to another cuff portion of the set of handcuffs. The connection portion can be a linked metal chain, a cable, a cord, or other suitable connector. The housing structure 110 is made of metal, hard plastic, or other suitable materials which are durable enough to be abused by the wearer without damaging the housing structure 110. Additionally, the housing structure 110 contains at least two sensor modules 120, a controller unit, a communication unit, and a power unit. In some embodiments, uncoupling the housing structure 110 from the set of handcuffs may provide access to the interior components comprised of the modules and units of the vitals monitoring handcuff apparatus 100. In further embodiments, the portion of the housing structure 110 that is obscured by the set of handcuffs may further comprise an access panel which shields the internal modules and units from the set of handcuffs. In even further embodiments, the access panel may comprise a plate portion and a locking mechanism that must be disengaged in order for law enforcement officials to access the internal modules and units for maintenance, updates, or repairs between detainments.

In some embodiments, the housing structure 110 is configured in a complimentary shape to the set of handle cuffs. The complimentary shape of the exterior of the housing structure 110 should have roughly the same silhouette, similar dimensions, and/or general shape of at least a portion of the set of handcuffs, while the interior of the housing structure 110 also has sufficient space for the modules and units. The complimentary shape of the housing structure 110 (namely the housing structure's 110 exterior) should include a wall that is flush (or nearly flush) with the inner circumference of the set of handcuffs which contacts the wearer's wrist during detainment and should have sufficient length along the set of handcuff's inner circumference so that a plurality of sensor modules 120 may contact and gather data from the wearer's wrist during detainment. In some embodiments, the complimentary shape may resemble and be the length of a double strand and/or check plates of a common set of handcuffs. The complimentary shape of the housing structure 110 should promote the contacting of the plurality of sensor modules 120 with the wearer's wrist while the set of handcuffs are in the locked position on the wearer.

Ideally, the complimentary shape of the housing structure 110 allows the edges of the housing structure 110 to be flush with at least part of the double strand and/or check plates to prevent snagging, tampering, damage, dust and liquid intrusion, etc. to the housing structure 110 and the interior components. Alternatively, the complimentary shape of the housing structure 110 may contact only one strand of the double strand portion of the set of handcuffs, so long as the plurality of sensor modules 120 retain sufficient contact with the wearer's wrist to perform their respective functions during detainment.

In alternative embodiments, the complimentary shape of the housing structure 110 may only comprise a small segment of the base and/or double strand of the set of handcuffs. This small segment may comprise as little as about one-fourth of the total circumference of the wearer's wrist.

In some embodiments, the present invention includes the plurality of sensor modules 120 protruding through to the exterior of the housing structure 110. The sensor modules 120 have an interior portion that is within the interior of the housing structure 110 and an exterior portion that protrudes through the housing structure's 110 exterior so that the sensor module 120 makes contact with the wearer's wrist during detainment. These sensor modules 120 may be one or more of the following: a photoplethysmography (“PPG”) sensor having one or more emitters and detectors; a bioimpedance sensor; a dermal temperature sensor; a blood pressure measuring system having a plurality of nodes; an serum pressure oxygen (commonly called a “SpO₂”) sensor; an electrocardiogram (“ECG”) sensor; an electrodermal activity (“EDA”) sensor; and an array of activity level modules comprised of one or more of an accelerometer, a gyroscope, a rotary encoder, a calorie measurement sensor, a temperature sensor, a dermal moisture measurement sensor, a displacement sensor, a pedometer, and an altimeter. The individual types of sensor modules 120 for taking biometric readings/vitals measurements may be configured as single sensors or as arrays of sensors.

In some embodiments, the sensor modules 120 of the vitals monitoring handcuff apparatus 100 are spaced throughout the portion of the housing structure 110 which makes contact with the wearer's wrist while the vitals monitoring handcuff apparatus 100 is in the locked position during detainment. Those skilled in the art will appreciate that a plurality of sensor modules 120 in such an arrangement as depicted in FIG. 1 will allow the various types of sensors to contact the wearer's wrist while the vitals monitoring handcuff apparatus 100 is not tightened to an undesirable and/or uncomfortable position on the wearer. In further embodiments, the sensor modules 120 are spaced out and configured around the portion of the housing structure 110 contacting the wearer's wrist when in the locked position in such a way that at least one sensor is always touching the wearer. In some embodiments, the controller unit contains a sensor module monitoring program which discerns whether a particular sensor module is touching the wearer due to the received data from the sensor module. For example, if the light emitted and detected by a PPG sensor system sends measurement data to the controller unit showing that the light reflected had too high or low a value, the sensor module monitoring program will instruct the controller unit to disregard the measurement data because it is outside an anticipated range that indicates that the measurement data was not collected while the PPG was making contact with the wearer's wrist. In some embodiments, the sensor modules may be configured to extend beyond the contact point on the housing structure where the sensor modules are affixed to the exterior of the housing structure. This extendable sensor module would allow the sensors to remain in contact with the wearer's skin without the need for the vitals monitoring handcuff apparatus to be too tightly ratchetted on the wearer. In this embodiment, the extendable sensor modules may be configured to extend or retract based on the wearer's wrist movements while the vitals monitoring handcuff apparatus is in the locked position.

In further embodiments, the extendable sensor modules further comprise spring mechanisms which control the extension and retraction of the extendable sensor modules. In these embodiments, the individual sensors may have attached structures that force them to protrude further beyond their resting, dormant, or inactivated position from the housing structure of the vitals monitoring handcuff apparatus after being activated.

In alternative embodiments, the sensor modules may be comprised of contactless sensors adapted to gather their readings either dermally or sub-dermally using known techniques and equipment such as non-contact infrared (or near infrared) sensors. The types of sensors typically send light towards the wearer and detect the reflected light to determine the amount of light absorbed by some part of the wearer, such as the wearer's hemoglobin in their veins and arteries absorbing infrared light instead of reflecting it. Another example of a contactless sensor module is a non-contact ECG sensor used in some medical settings, including in-treatment center and remote (at an out-patient center or at the patient's home). Yet another example of a contactless sensor includes contactless PPG sensors (i.e. an optical pulse oximeter) which may be adapted to pulsate light over a large area of skin in order to record the reflected light to determine a biometric and/or vitals measurement (collectively, just “vitals measurement”). This PPG sensor may not rely on a specific controlled light path through the skin area but rather a wider area and smaller depth.

In some embodiments, the sensor modules 120 are controlled by the controller unit which has a processor, a computer memory storage unit, a volatile memory unit, and circuitry connecting the controller unit to the modules and units within the housing structure 110. The controller unit may be programmed with an operating system and individual programs which control the operation of the modules and units, and calculates the wearer's vital signs by inputting the data from the sensor modules 120 into corresponding software. The controller unit may send an output from the calculating software to a communication unit that will display the wearer's vitals measurements and/or alert the law enforcement official of an emerging or active medical condition of the wearer. Vitals measurements are a form of biometric data. The communication unit can do one or more of the following: display vitals measurements (for example, using a LCD or LED screen 150), play an alert signal through a speaker in the housing structure 110, and send a wireless signal (for example: Wi-Fi™, wireless mobile network, or Bluetooth™) to a program or device that is configured to display data to or alert law enforcement officials of a medical issue of the wearer. Example medical issues and/or events include cardiovascular events such as palpitations, hypertension, or a heart attack; respiratory events such as a lack of oxygen or hyperventilation; and skeletal and/or muscular problems causing pain and/or panic; and similarly suited medical issues causing a need for immediate or relatively soon medical aid or intervention.

One embodiment of the communication unit includes a cellular chip that sends a wireless signal to a receiver or network such as a mobile telephone carrier network or a satellite network. This wireless signal can then be relayed to law enforcement officials on-site or at a designated location. Another embodiment of the communication unit includes a Bluetooth™ chip that sends the wireless signal to a Bluetooth™ receiving device belonging to the law enforcement official, whether on his or her person or to a unit in his or her vehicle.

A PPG sensor operates by at least one emitter emitting an amount of light. The light is reflected by a vein in the wearer's wrist. The reflected light is measured by at least one detector. The emitter is controlled by the controller unit and the detector sends a measurement of the amount of light reflected by the vein to the controller unit. The controller unit inputs the measurement of emitted light and measurement of detected light into a heartrate calculation formula (for example and overly simple: number of detected heartbeats detected by the sensor over a determined number of seconds and output as beats per minute (“BPM”)) which calculates the heartrate of the wearer to some degree of accuracy. Additionally, current technology and software are able to estimate the wearer's respiration rate based on the emitted and detector-measured light.

A bioimpedance sensor operates by sending an electrical current through at least one electrode to at least one other electrode. The amount of electrical current sent through the first electrode is instructed by the controller unit. And the data for the amount of current received by the second electrode is sent to the controller unit. In some embodiments, the controller unit then processes the two measurements into calculation software to calculate the wearer's respiration rate. Bioimpedance sensors have other potential uses for the vitals monitoring handcuff apparatus 100 that those skilled in the art may appreciate such as calculating skin-water content. An array of bioimpedance sensors on the housing structure 110 provides an appreciable benefit to the vitals monitoring handcuff apparatus 100 because individual wearers will have different wrist sizes, bony anatomy, skin-water content, vascular branch size and locations, and ratios of adipose, skin, bone, and muscle content within the array of sensor modules 120. This array of bioimpedance sensors will help ensure the collection and accuracy of data collected and provided to the controller unit.

A dermal temperature sensor measures and sends the measurement of a wearer's skin temperature at a contact point to the controller unit. The controller unit uses this data to calculate the wearer's body temperature. Additionally, an ambient temperature sensor may send a measurement of the ambient temperature to the controller unit so that the controller unit may use these those measurements in a formula to more accurately calculate the wearer's body temperature. Alternatively, the controller unit may connect to the internet via a wireless connection and retrieve the reported ambient temperature. The controller unit engages a GPS module to determine the location of the wearer. This engagement and use of the GPS module can serve many purposes. Particularly, a GPS module within the vitals monitoring handcuff apparatus 100 allows law enforcement officials to locate the wearer. This is appreciably useful in the event that the wearer is lost or has fled detainment. Additionally, the controller unit may retrieve the wearer's location using the GPS module in order to determine the ambient temperature at the wearer's location via the internet. The controller unit may use this GPS-located and retrieved ambient temperature measurement in a calculation formula with the dermal temperature sensor to more accurately calculate the wearer's body temperature.

A blood pressure measurement system having a plurality of nodes and an inflation system may be used to measure a wearer's blood pressure. The blood pressure measurement system operates by receiving a signal from the controller unit to inflate the inflation system until blood flow through the veins in the wearer's wrist stops. Then, the nodes detect once the blood in the veins in the wearer's wrist begins to flow again at both a systolic pressure and normal blood flow pressure. These measurements are sent to the controller unit which calculates the wearer's blood pressure.

A SpO₂ sensor having a plurality of nodes with at least one emitter and at least one detector may be used to measure the amount of oxygen in the wearer's blood. The controller unit causes at least one emitter of the SpO₂ sensor to emit an amount of light. The light is reflected by the blood in the wearer's veins. The light is measured in both the red and infrared spectrums. At least one detector measures the reflected red and infrared light and sends this data to the controller unit which inputs the data for the amount of emitted light and the amount of measured reflected light into an formula which calculates a measurement of the wearer's blood-oxygen levels.

An ECG sensor measures the small amount of electrical current produced by the action of the wearer's heart pumping blood through the wearer's veins. The ECG sensor sends this data to the controller unit which uses the measurement with a formula to calculate the wearer's heart's rhythm and electrical activity.

An EDA sensor measures the changes in electrical activity resulting from changes in the wearer's sweat glands. Changes in measurements of the electrical activity may indicate changes in the stress level of the wearer. The EDA sensor sends its measurement data to the controller unit which may use that data in corresponding formula and/or software to calculate a stress level of the wearer.

An array of activity level modules comprised of one or more of an accelerometer, a gyroscope, a rotary encoder, a calorie measurement sensor, a temperature sensor, a dermal moisture measurement sensor, a displacement sensor, a pedometer, and an altimeter may be present in the vitals monitoring handcuff apparatus 100 to measure an activity level of the wearer. In some embodiments, it is beneficial to include multiple of the same activity level module components (such as three gyroscopes with each at different orientations) to more accurately calculate activity levels. Where necessary, the controller unit will send instructions to the appropriate activity level modules in order for the activity level modules to collect a measurement of the wearer's activity level. Whether instructed by the controller unit or passively measured by the activity level modules, the data collected by the activity level modules is relayed to the controller unit which inputs the data into corresponding activity formulas to calculate the wearer's activity level.

In some embodiments, an access point 160 to a functionality button is present on the housing structure 110. The functionality button may turn the present invention on or off, and it may allow the present invention to enter a “silenced” mode. The functionality button may be configured where a “long press” (applying pressure to activate the button over several seconds) turns the present invention on or off, and “short presses” turns the “silenced” mode on or off. The access point 160 may be configured so that it is so small that a tool (such as a SIM tray ejector tool for mobile phones) is required to interact with the functionality button.

In some embodiments, the vitals monitoring handcuff apparatus 100 may remain in a dormant state awaiting activation to an activated state by a gesture from the law enforcement official or when the controller unit receives a sufficient amount or type of data from one or more of the sensor modules 120. In further embodiments, while in the dormant state, some processes (such as wireless, Wi-Fi™, and Bluetooth™ connectivity, illuminating the display unit, or the active collection of biometric data) are disabled to reduce the amount of power the device latently drains from the power unit. In further embodiments, the vitals monitoring handcuff apparatus 100 remains in the dormant state so long as the set of handcuffs are not ratchetted in a closed position on the wearer. An example of the closed position is when the set of handcuffs are secured around the wearer's wrists so that the wearer cannot remove the handcuffs. In such an embodiment, the vitals monitoring handcuff apparatus 100 includes a sensor that detects whether the set of handcuffs are ratchetted to the closed position on the wearer.

The aforementioned gesture, for example, may be in the form of briefly shaking the device to cause the activity level modules (such as the accelerometer(s), gyroscope(s), or displacement sensor) to produce data to send to the controller unit. The controller unit, in response to the receipt of data from the activity level modules, then switches the device into the activated state and attempts to collect biometric data from the sensor modules and relay that data by transmitting the data via the communication unit and/or displaying the data via the display unit.

The aforementioned sufficient amount or type of data to switch the device from the dormant state to the activated state may be predetermined (and changeable) by a program contained on the controller unit. The controller unit, in response to the receipt of the sufficient amount or type of data, then switches the device into the activated state and attempts to collect biometric data from the sensor modules and relay that data by transmitting the data via the communication unit and/or displaying the data via the display unit.

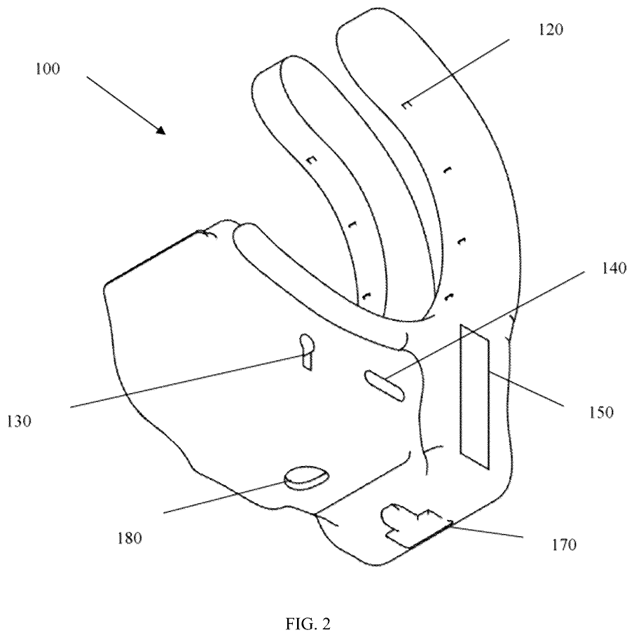

Referring to FIG. 2. In some embodiments, a power unit is located within the housing structure 110 and provides power to the other units and modules of the vitals monitoring handcuff apparatus 100. In further embodiments, the housing structure 110 is adapted to have a port 170 which couples with a power subsystem to charge the power unit within the housing structure 110. It should be appreciated by those skilled in the art that the port and power sub may be comprised of, in one embodiment, a multipurpose data-transfer and charging connection such as a USB charging connection. The USB charging connection can connect with the power unit to charge the vitals monitoring handcuff apparatus 100 and to the controller unit as an access point which could be used to transfer data to and from the controller unit to an external source such as a computer.

In some embodiments the housing structure's 110 port 170 for the power unit further comprises a cover (such as a rubber-coated panel or gasket between the housing structure 110 and the cover panel) to prevent dust and damage of the power unit's port 170. The cover for the port 170 may also further comprise a locking mechanism such as a magnetic lock, keyhole lock, or electronic lock, for the purpose of preventing tampering with the port 170 on the housing structure 110 of the vitals monitoring handcuff apparatus 100.

In some embodiments a Bluetooth™ pairing button can be included on the housing structure 110. In further embodiments, the Bluetooth™ pairing button is located in an interior wall of the power unit's port 170. The Bluetooth™ pairing button in the interior wall of the power unit's port 170 is located in such a way that it does not conflict with the aforementioned cover of the port 170 nor does it conflict with the charging system within the port 170. Law enforcement officials may access and use the Bluetooth™ pairing button to pair the vitals monitoring handcuff apparatus 100 to a device such as a cell phone or computer.

In some embodiments, a connection hole 180 in the housing structure 110 is present to allow the connection portion of a set of handcuffs to connect one cuff to the other cuff. This connection hole 180 can be narrow enough to only allow the connection portion of the set of handcuffs to fit through the connection hole 180. In such an embodiment, one cuff of the set of handcuffs may need to be disconnected from the connection portion of the set of handcuffs and then reconnected once the cuff is coupled to the housing structure 110. In other embodiments, the housing structure 110 may have a connection hole 180 that is large enough for the cuff of the set of handcuffs to be moved through the connection hole 180 before coupling with the housing structure. In further embodiments, a housing structure 110 with a connection hole 180 that is large enough for a cuff of a set of handcuffs to move through the connection hole 180 may further comprise a locking mechanism that prevents the cuff of the set of handcuffs from being undesirably moved backwards through the connection hole 180.

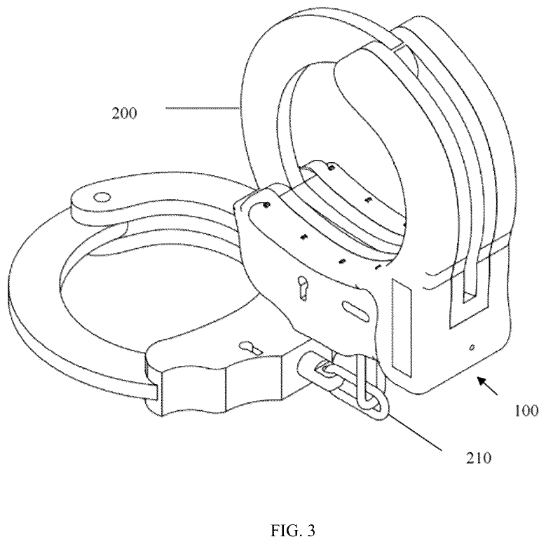

FIG. 3. Illustrates an embodiment of the present invention wherein the housing structure 110 is coupled to a standard set of handcuffs 200 (such as ASP handcuffs). One skilled in the art should find it appreciable that the housing structure 110 may be constructed to have a complimentary shape to the set of handcuffs (as depicted in FIG. 3), provides access to the set of handcuffs' 200 keyhole at the keyhole access point 130, and to the set of handcuffs' 200 secondary locking mechanism at the secondary access point 140. As shown in FIG. 3, the complimentary shape of the housing structure 110 should be constructed to resemble at least a portion of the set of handcuffs coupled with the housing structure 110. The embodiment of FIG. 3 also shows a chain link connection 210 between one cuff and the other cuff of the set of handcuffs 200. This chain link connection 210 passes through the connection hole 180 of the housing structure 110. In the illustrated embodiment, the connection hole 180 is not large enough for the cuff of the set of handcuffs to pass through; therefore, this embodiment requires the chain link connection 210 to be attached to the cuff after the cuff has coupled with the housing structure 110.

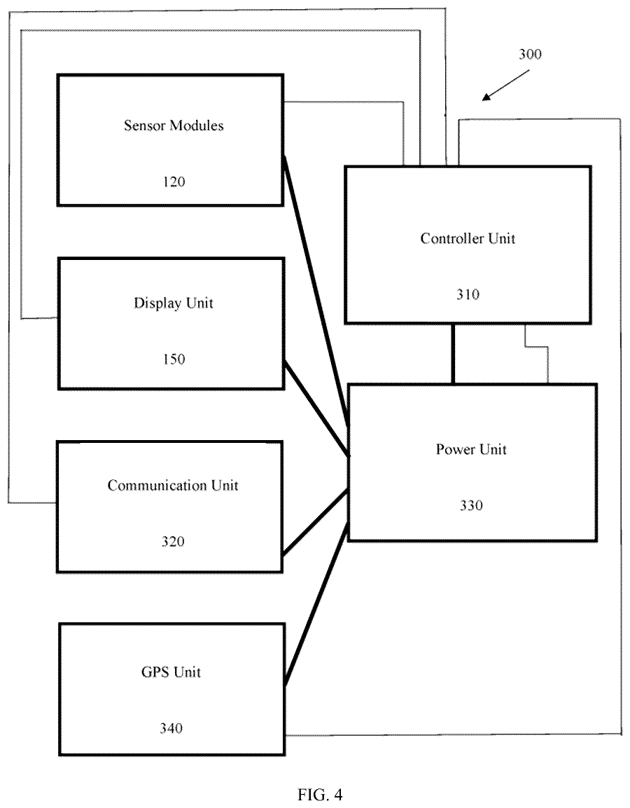

FIG. 4 shows, in some embodiments of the present invention, a configuration of the components within the housing structure 110 of the vitals monitoring handcuff apparatus 100. Sensor modules 120, the display unit 150, the communication unit 320, GPS unit 340, and the controller unit 310 are all connected to and receive electrical power from the power unit 330. The power unit 330 subsequently includes a battery to hold an amount of electrical charge. The battery is charged and recharged by a power subsystem. The power subsystem may comprise known technologies such as a charging port, insertable battery cells, inductive charging (aka wireless or cordless charging), or any other charging system appreciated by those skilled in the art. In some embodiments, the insertable battery cells are also rechargeable by way of an exterior charging station or similarly suited method. In other embodiments, the inductive charging is accomplished by the power unit comprising a battery connected to an inductive charging receiver and that receiver being charged by an inductive charging receptacle that the vitals monitoring handcuff apparatus may be placed on to initiate charging the battery within the power unit. In further embodiments, the inductive charging battery, receiver, and charging receptacle use either tightly-coupled electromagnetic inductive charging (also known as non-radiative charging) or radiative electromagnetic charging. In even further embodiments, the vitals monitoring handcuff apparatus has an inductive charging station designed to hold and charge the vitals monitoring handcuff apparatus. Those skilled in the art will appreciate that, in some embodiments, the vitals monitoring handcuff apparatus 100 will remain usable to restrain the wearer even when the power unit is discharged and cannot power the electrical components of the present invention.

In some embodiments of the present invention, the sensor modules 120, display unit 150, communication unit 320, GPS unit 340, and power unit 330 are controlled by a controller unit 320. The controller unit 320 comprises a processor, a computer memory storage unit, a volatile memory unit, and circuitry. The controller unit 320 may comprise various executable programs used to control the modules and units, additional programs which calculate biometric data based on the data received from the sensor modules, an operating system, and other suitable executables which are necessary for the function of the vitals monitoring handcuff apparatus 100. The controller unit 320 sends instructions to, receives data from, and processes outputs for the other modules and units of the vitals monitoring handcuff apparatus 100.

The sensor modules may constantly record and relay the data generated for the wearer to the controller unit, even when a medical issue or event is not actively occurring. This continuous monitoring of the wearer's vitals measurements allows for a baseline of the wearer's biometric and/or vitals measurements to be determined and better allows the controller unit to determine that the wearer is experiencing a medical event or issue. Put another way, through continuous monitoring, the controller unit may be able to accurately determine the emergence of a medical issue or event earlier than if no active monitoring and tracking were taking place for the wearer's vitals measurements. Ideally, the wearer's baseline vitals measurements are detectable and ascertained by the present invention within a small window of time from the moment the vitals monitoring handcuff apparatus is placed on the wearer. The earlier this baseline is established, the more effective the present invention will be at identifying an emerging or present medical event, issue, or problem.

Data recorded by the sensor modules may be entirely recorded by the vitals monitoring handcuff apparatus or data may be recorded based on changes in measurements (such as a spike in heart rate) or at set intervals of time.

Due to the nature of continuous monitoring and recording of generated data, the controller unit should have access to sufficient computer readable and writable memory to record, format, and tabularize the data. Additional data should also be recorded for categorization and later searching. Therefore, the controller unit will need to run some form of generic (such as recording the sensor data and metadata (such as timestamps, dates, locations that measurements were taken, etc.) into a simple table) or specialized software to create a file, record data generated by the sensor modules, track trends in that data, and be retrievable for later analysis or use. The metadata may be necessary for identification of the correct sensor data within a generated log or log file of the measured biometric data/vitals measurements. Timestamping vitals measurements may also be required for enabling the controller unit or a centralized processor to identify and/or calculate trends of vitals measurements in order to identify anticipated medical issues or emergencies.

Recorded data may be tied to a specific wearer (such as the detained) or it may simply be associated with the activation time of the vitals monitoring handcuff apparatus. For example, a profile may be generated having activation time data for the vitals monitoring handcuff apparatus, and that activation time may be the used to identify the recorded vitals measurements, medical issues or events, or other data generated by the wearer (such as location tracking). The profile may be assignable to a specific wearer or merely be generated with each activation or event of the vitals monitoring handcuff apparatus and simply identified (such as the activation time being the profile name). The profile may be assigned an identifier (such as the timestamp for activation, a sequential number, the name of the wearer, etc.) for organization and retrieval of specific profiles and logs. The recorded data, including data tied to each profile, may be stored in log files or logs within the device for later retrieval or transmission. The log files may be created when the vitals monitoring handcuff apparatus is activated, removed from its holster or holder, or upon the press of a button or other signal from another device at the law enforcement official's input. Further, the vitals monitoring handcuff apparatus or other device that the log files may be created or stored on may create a divide or new file upon certain events such as: when the handcuffs are removed from the wearer, when the sensors do not receive data over a set interval, when the vitals monitoring handcuff apparatus is placed in an inactive or rest mode, when the vitals monitoring handcuff apparatus is otherwise deactivated or put into a rest mode, or other instance where data may no longer be being received by the sensors.

The recorded data may be transmitted either wired or wirelessly to a computer or other personal device (such as a cell phone). This data may then be uploaded to a server for longer term storage and tracking, group analysis, presentation to a third party, or for other contemplated reason as is necessitated by the law enforcement context of the devices use and the gathering of said recorded data. In particular, recorded data is very useful for analysis of the wearer's vitals/biometric measurements and potential medical issues when examining a situation where the medical issue caused harm to the wearer (including up to death). This recorded data may be beneficial for law enforcement officials to help identify medically fragile detained persons whom the law enforcement official could not assess the cause of a medical issue. This may aid the law enforcement official and governing body in assigning liability for the occurrence of a medical issue during the wearer's detainment.

In addition to the time of activation and time of vitals/biometric readings, and the vitals/biometric readings themselves, the vitals monitoring handcuff apparatus may record broadcast alerts, diagnostics for the device, detected hardware and/or software errors, and times for all of the foregoing.

A companion application, accessible either through the internet or on standalone application on a personal device (such as a cell phone or computer) or piece of law enforcement equipment (like the terminal in a squad car), may allow the law enforcement official or their governing body to quickly access the logs from the vital monitoring handcuff apparatus. In some embodiments, the log files themselves may be recorded and stored on the companion application on the personal device instead of (or in addition to) the vitals monitoring handcuff apparatus.

In some embodiments, the companion application may have a user account sign on for the law enforcement official and/or their department or governing body. Such an account sign-on and verification protocol is necessary because of the sensitive nature of the situations and contexts associated with the use of the present invention. Once logged in, the law enforcement official may access details about the handcuffs, log files from the handcuffs, current vitals measurements of the wearer, alerts or statuses, or adjust settings for the vitals monitoring handcuff apparatus or a specialized external device, and so forth. In further embodiments, the companion application may present the baseline biometric/vitals measurements of the wearer and wherein the companion application may be suited for better determining whether a medical issue, event, or situation is emerging or present.

Data recorded by the present invention may be displayed as a timeline within the companion application so that the law enforcement official, governing body, or third party may see when a medical event, issue, or situation occurred or what the wearer's biometric/vitals measurement was at a specific interval. Ideally, the companion application should allow the law enforcement official to perform at least one of the following actions through interaction with a user interface in the companion application: review the wearer's current vitals measurements, review trending vitals measurements, review maximum recorded vitals measurements, display the status of the vitals monitoring apparatus, review a log file created once the vitals monitoring apparatus was activated and the handcuffs engaged in the locked position on the wearer for detainment, add notes to the log file, and transmit data to a centralized server.

In further embodiments, the law enforcement official may be able to add notes to the log file on the companion application to add context to the arrest or vitals measurements. To clarify, the addition of notes does not mean that the law enforcement official will have access to the biometric/vitals measurements with the ability to add, edit, or delete any recorded data. Any log ins, access, notes, or other interaction with the log file or companion application should be reviewable by a centralized administrative user account to maintain oversight due to the nature of the use of the present invention.

In alternative embodiments, the company application may be accessed through a website in addition to, or instead of, being downloaded on the law enforcement official's device(s) or other equipment.

In other exemplary embodiments of a vitals monitoring handcuff apparatus 100, the housing structure may be constructed onto and permanently affixed to a set of handcuffs. Those skilled in the art will appreciate that the nature of the permanently affixed housing structure is more difficult for a wearer or other individual to tamper with or damage. Additionally, some users may find it appreciable to manufacturing and cost benefits in permanently affixing the housing structure to a set of handcuffs.

In some embodiments of the present invention, a law enforcement official sets the vitals monitoring handcuff apparatus 100 to an “on” mode so that the modules and units are operating and prepared to measure the wearer's vitals measurements and alert the law enforcement official of the wearer's potential medical issues. Next, the law enforcement official takes the vitals monitoring handcuff apparatus 100 (that is coupled to the set of handcuffs) and places the device onto the wearer who is being placed under detainment. The controller unit causes the sensor modules to collect measurements and data, which is relayed back to the controller unit for processing during the detainment. Upon detection of vitals measurements outside of given parameters, the controller unit will cause the communication unit to display the vitals measurements, play an alarm to alert the law enforcement official, and/or transfer the measurements and/or alarm to an external receiver such as an app on a smartphone or computer that may display the vitals measurements or alert the law enforcement official of any. The law enforcement official may place the device into its “silent” mode once the alert has sounded so that the alter does not continue to sound as the law enforcement official determines and executes an appropriate course of action. Finally, after the vitals monitoring handcuff apparatus 100 has been removed from the wearer, the law enforcement official may recharge the device by connecting it, at the port, to a power subsystem.

The given parameters may be a threshold event such as a heartrate over 160, a respiration rate over 120, a blood pressure reading with a systolic number over 200 and/or a diastolic number over 120, an irregular ECG or EKG reading, and/or combinations thereof. In further embodiments, the given parameters may be a trending event and analyzed by the processor of the controller unit to identify a trending change in any of the biometric/vitals measurements over a set interval in order to better alert the law enforcement official of a possible oncoming medical condition, issue, or emergency of the wearer.

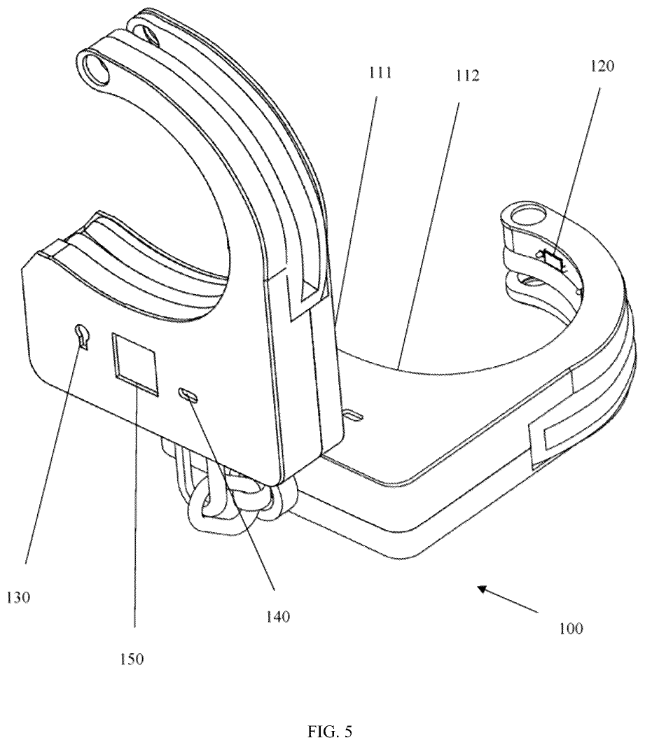

FIG. 5 shows, in some alternative embodiments of the present invention, that the vitals monitoring handcuff apparatus 100 may include two housing structures, each housing structure (represented with housing structure A 111 and housing structure B 112) being affixed to a separate cuff of a set of handcuffs. These housing structures serve as the base structure for components identified and described in previous embodiments of the present invention including, but not limited to, the sensor modules 120, the keyhole access point 130, the secondary access point 140, and the LCD or LED screen 150. Alternatively, in other embodiments, the vitals monitoring handcuff apparatus may be built onto or as an original part of each cuff of the set of handcuffs. Those skilled in the art will appreciate that this embodiment with the housing structure and associated circuitry, sensors, controller unit, communication unit, and power supply will be able to collect data from both of the wearer's wrists. These additional sensor modules (which results in more contact points with the wearer's skin) and the additional amount of data from those sensors will provide law enforcement officials more continuous and accurate data than embodiments of the vitals monitoring handcuff apparatus that only make contact with one of the wearer's wrists.

In further embodiments of the present invention, one housing structure, for example housing structure A 111, contains the controller unit and the communication unit while the other housing structure, for example housing structure B 112, contains the power unit include a battery. In these embodiments, the units and modules housing within both housing structures are connected via a channel or cable which may also serve as the connection for the two cuffs of the set of handcuffs. This cable may have an interior cable portion where wires and other means could connect the units and modules within one housing structure to the units and modules within the other housing structure. Those experience in the art will appreciate that such a cable would benefit from a swivel connection at the base of each housing structure which would allow the cable to rotate around the connection and be resistant to being manipulated into a loop or undesirably bound position that could damage the cable or the coupling of the cable to either cuff.

In some embodiments of the present invention, each housing structure on the cuff of the set of handcuffs contains the essential elements of the invention so that each housing structure and their associated modules and units may independently operate without power, instruction, or other resource from any units within the housing structure couple to the other cuff. For example, the housing structure on each cuff of the set of handcuffs would include a first sensor module, a second sensor module, a controller unit, a communication unit, and a power unit. In further embodiments, the controller unit within one housing structure on the cuff of the set of handcuffs is configured to communicate with the controller unit within the other housing structure. This communication may be via their respective communication units and may use a short-distance form of electronic communication such as Bluetooth™ with proper encryption protocols to ensure device security. Such embodiments are useful for the controller units to share computation workload or for one controller unit to provide the other with vitals/biometric measurements so that the aggregate readings from both cuffs may be used for the calculation of vitals measurements to determine whether a medical issue or event is emerging or present. Additionally, the communication unit on each cuff may send its respective data to an exterior device (such as the law enforcement official's cell phone, computer, or other device) or network (such as a cellular or satellite network).

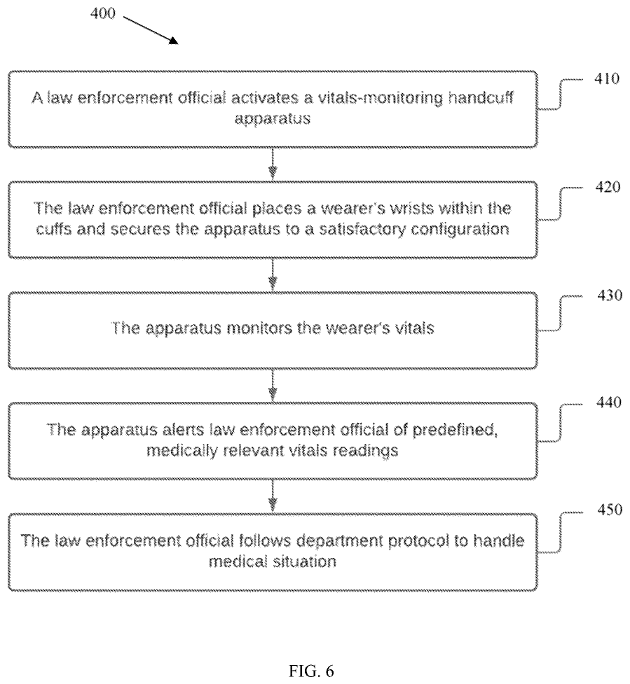

FIG. 6 discloses, in some embodiments of the present invention, a method for using a vitals monitoring handcuff apparatus. The method 400 includes a law enforcement official, hereafter as described in the method “user,” activating on a wearer's wrists and securing the cuffs to a satisfactory configuration 420; the vitals monitoring handcuff apparatus taking the wearer's vitals measurements using at least two sensors and a controller unit 430; a communication of the vitals monitoring handcuff apparatus alerting the user of predefined, medically relevant vitals readings of the wearer 440; and the user following appropriate protocols to handle the wearer's medical situation 450. The vitals monitoring handcuff apparatus may be retried from a case or charger, from a law enforcement official's belt, or other suitable storage container or location. Then, the user will activate the vitals monitoring handcuff apparatus so that the device may begin taking vitals measurements using the sensor modules. Some embodiments of the present invention may be configured to allow the user to select which types of vitals measurements are taken and include other settings such as the frequency of recordation of vitals measurements. Securing the cuffs to a satisfactory configuration (i.e. tightness) on the wearer includes ratchetting the teeth of each cuff until it is firmly secured to a desired tightness on the wearer's wrist. Alternatively, securing each cuff to a satisfactory configuration includes other forms of restraint such as pulling the cable taught in a set of zip-tie restraints. The controller unit monitors the vitals measurements from the sensor modules to determine if a medically relevant situation, issue, or emergency occurs. This monitoring of vitals measurements may be continuous at settable intervals (ex: every second, every quart of a second, every three seconds, etc.). If the wearer has a medical event which demands the user's attention, the cuffs will alert the user via one of the communication units methods such as a speaker, LED flash, alert sent to an external speaker or device, or other method which notifies the user of the wearer's medical event. Many users will then proceed to follow prescribed protocols and procedures that the user is trained to perform to handle the medical event, situation, issue, or emergency. These protocols and procedures may include examining the wearer, checking the vitals monitoring handcuff apparatus for a false alarm, and/or calling medical services so that the wearer can receive medical care from someone with more training.

In some embodiments, the present invention is presented as a system including a vitals monitoring handcuff apparatus and a specialized external device. The specialized external device may be a wearable, accessory, computer or in-vehicle terminal, or other piece of equipment located on the law enforcement official or in their immediate area (such as attached to their squad car). The specialized external device may be a device that is clippable, clampable, or otherwise configured to attach (collectively “device attachment structure”) to the law enforcement official's belt, uniform, or other equipment. The device may include a speaker and be able to be connected with the law enforcement official's body camera, cell phone, pager, walkie talkie, or another, custom communication device, a computer or terminal located within or on the law enforcement official's vehicle, a centralized (usually stationary) system, computer, or server (for example, at the law enforcement official's precinct), or other device capable of alerting the law enforcement official of the wearer's emerging or pending medical event, issue, situation, or emergency. Regardless of the form of the specialized external device, the goal is to first inform the law enforcement official on the scene with the detained wearer(s) medical issue or emergency, and, secondly, inform other bodies such as emergency medical services or the law enforcement official's governing body.

Further, in addition to the processors, memory, circuitry, and power unit necessary for computer function, the specialized external device may include onboard storage (such as an onboard SSD or SD card), a vitals monitoring handcuff apparatus connection method (such as Bluetooth™, Near-Field Communication, Zigbee, Z-Wave, Ultra-Wideband, and equivalents (with corresponding hardware) to operably connect with the vitals monitoring handcuff apparatus), a cellular chip or wireless chip for connecting with a wireless network for wireless data transmission, an input method (such as a numpad, small keypad, or series of buttons tied to specific functions), an apparatus for charging the vitals monitoring handcuff apparatus when they are stored on the specialized external device, an apparatus holster structure for the vitals monitoring handcuff apparatus, and an emergency call button which notifies emergency services of a need for them (likely related to the condition on the wearer). The emergency call button may be included as one of the buttons of the input method.

As a charger for the vitals monitoring handcuff apparatus, the specialized external device may provide an apparatus charger comprising a wired connection (such as a cable with a male piece to connect with the vitals monitoring apparatus's female receiver) or a wireless charging connection (such as those commonly used for cellular phones including metal contacts and induction charging).

The specialized external device may include a GPS tool (which may track through common GPS navigation methods such as when connected to a wireless or cellular network or connected via a passthrough (such as Bluetooth™ to the law enforcement official's other equipment or vehicle's equipment).

The specialized external device may include an LED or LED display, or other durable screen, for displaying the wearer's vitals measurements or a visual alert for indicating the wearer's condition (such as if the system detects that the wearer is experiencing a medical event or emergency). The display may also display error codes, transmission status, connectivity errors, etc. Ideally, the specialized external device is configured to simultaneously send audio and visual indications of the wearer's medical issue in order to most effectively grab the attention of law enforcement personnel on the scene.

Embodiments of the vitals monitoring handcuff apparatus that do not feature a speaker or LED/LCD screen are likely to be more durable and tamperproof and, therefore, desirable in some law enforcement contexts where detained wearers are more likely to seek to damage the vitals monitoring handcuff apparatus.

In embodiments of this system including the specialized external device, the vitals monitoring handcuff apparatus may send recorded data related to the wearer's vitals measurements and/or medical events or an alarm or alert signaling a medical issue so that the specialized external device may alert the law enforcement official, medical assistance, or the law enforcement official's governing body of the status of the wearer. This alarm or alert may also activate additional system

In some embodiments, the specialized external device may serve as an intermediary to receive data from the vitals monitoring handcuff apparatus and forward it to a centralized system, server, or computer, or review center. This centralized system may be where the governing body of the law enforcement official is located, in a cloud server, or at some other location. For record keeping purposes and due to the sensitive nature of the wearer's biometric/vitals measurements, any centralized system should include some sort of backup for the data.

The recorded data sent from the vitals monitoring handcuff apparatus or specialized external device may be used in conjunction with other law enforcement data to identify trends and emerging issues of wearers. For example, if multiple devices are sending data identifying an emerging medical issue of multiple detained wearers (such as lowered O₂ readings, elevated temperature, and increased skin hydration), the law enforcement officials/governing bodies may be able to get in front of a virus or disease emerging and provide preventative or early acting medical care.

In some embodiments, the specialized external device may couple with the vitals monitoring handcuff apparatus. Further, the specialized external device may include a battery which may provide charge to the power unit of the vitals monitoring handcuff apparatus while coupled or “holstered.” Even further, removing the vitals monitoring handcuff apparatus from the specialized external device, having an apparatus holster structure to act as a holster or holder for the vitals monitoring handcuff apparatus, may activate the vitals monitoring handcuff apparatus to its “on” status where it is ready to measure and transmit data related to the wearer's vitals measurements.

Some embodiments of the vitals monitoring handcuff apparatus may be adapted for long term wear and biometric recordation. Rather than short detainment, these embodiments may focus on the jailed or incarcerated. The purpose of the vitals monitoring handcuff apparatus would remain the same, providing an alarm or alert to law enforcement, correction, detention, or other officials of an emerging or present medical event, issue, situation, or emergency of the wearer. Speakers, flashing LEDs, or on-device communication means may alert officials or other incarcerated persons of the wearer's condition or issue while the communication device may also wirelessly transmit the vitals/biometric measurements to a centralized system (such as a computer or server) adapted to store the recorded data to profiles that are assigned to either the specific vitals monitoring handcuff device or the wearer. These longer-term solutions may be in the form of a cuff or band attached to the wearer.

In some embodiments, the vitals monitoring handcuff apparatus, directly or through an external device such as a specialized external device or the law enforcement official's personal device or another device such as the vehicle's terminal and/or speaker system, may provide an alert or alarm if the plurality of sensor modules do not retain sufficient contact over time with the wearer to accurately record the wearer's vitals measurements and/or create a baseline.

In some embodiments, each vitals monitoring handcuff apparatus and/or specialized external device may include a serial number or other unique identified which more than one may be assignable to a specific law enforcement official. This assignment of multiple vitals monitoring handcuff apparatuses and a specialized external device allows for quick pairing and seamless swapping or transitioning of different sets of cuffs on the same wearer. Pairing one of these devices with a wearer may be performed manually with something as simple as pressing a Bluetooth™ pairing button on a set of vitals monitoring handcuffs and a device on the law enforcement official or with simple means such as Remote Function Call (“FRC”) communication between the law enforcement official's other gear such as a device on their belt (such as the specialized external device) or their body cam or communication device and the vitals monitoring handcuff apparatus. In the event that a single law enforcement official carries multiple vitals monitoring handcuff apparatuses (as may be common)

If profiles are tailored not to the event but rather to the wearer, then the vitals monitoring handcuff apparatus system should include options to quickly identify the wearer so as to link their previous data with their presently collecting data. This may be accomplished as simply as assigning an input-able identifier into the vitals monitoring handcuff apparatus so that the system may seamlessly link the wearer to the wearer's profile. For example, the vitals monitoring handcuff apparatus may have a small keypad under an access panel (potentially lockable) where the law enforcement official may assign a 5-digit number that is tied to the wearer's profile within the centralized system.

In some embodiments of the aforementioned system including a vitals monitoring handcuff apparatus and a specialized external device, the use of the system generally entails the steps of (in flexible order): unboxing the vitals monitoring handcuff apparatus and specialized external device; going through a first time activation setup wherein a companion application may be downloaded on the law enforcement official's cell phone or other computerized device and used to registered the vitals monitoring handcuff apparatus and/or specialized external device; pairing the vitals monitoring handcuff apparatus and/or specialized external device to one another and/or the companion application via a wireless method (such as via Bluetooth™) or a wired method (such as a specialized or USB cable connection); the law enforcement official clamping the specialized external device to the law enforcement official's belt and holstering the vitals monitoring handcuff apparatus with an apparatus holster structure on the specialized external device; removing the vitals monitoring handcuff apparatus from the specialized external device's holster, activating the vitals monitoring handcuff to start receiving data from the sensors and creating a file (on the vitals monitoring handcuff apparatus or the specialized external device) tied to the time of activation; placing the vitals monitoring handcuff apparatus on the detained wearer; the sensors sending the wearer's biometric/vitals measurements to the controller unit on the vitals monitoring handcuff apparatus which may relay the data (via the communication unit) to the specialized external device to be recorded within the file; upon detection of vitals measurements outside of given parameters or when the processor anticipates an emerging medical issue (such as by detecting a progressively increasing heart rate over a short span of time) visually and/or auditorily alerting the law enforcement official of the wearer's condition (such as by a speaker or flashing LED on the vitals monitoring handcuff apparatus and/or the specialized external device); the law enforcement official going on about his or her business which caused the need for detaining the wearer; removing the vitals monitoring handcuff apparatus from the wearer; the vitals monitoring handcuff apparatus going to a rest or sleep mode or becoming deactivated upon not receiving acceptable (data that could be given by a human's contact) sensor data for a prescribed interval or when the vitals monitoring handcuff apparatus is holstered with the specialized external device; optionally, the law enforcement official adding notes to the log file; and the completed (due to no longer receiving data) log file being uploaded to a server.

In some embodiments, the specialized external device may include a plurality of devices working in tandem or concurrently to record the data sent via the vitals monitoring apparatus or one of the other specialized external devices. The specialized external device may also communication with the companion application. This redundancy in data recordation may sometimes be desirable due to the stressful nature of some forms of detainment of the wearer of the vitals monitoring handcuff apparatus and the law enforcement official.

It should be understood that the term law enforcement official may be referred to in the singular or the plural but will have the same meaning. It should also be understood that law enforcement official or officials are interchangeable with “user” if the device is used by another individual who is not a part of any body of law enforcement.

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A vitals monitoring apparatus coupled with a set of handcuffs comprising:
a housing structure having an exterior and interior, adapted to be coupled and uncoupled with the set of handcuffs used by a law enforcement official for detainment of a wearer, the interior of the housing structure containing a plurality of modules and units, the exterior of the housing structure constructed to generally have a complimentary shape to the set of handcuffs, the exterior of the housing structure configured to contact the wearer's wrist while the set of handcuffs is engaged in a locked position on the wearer's wrist during detainment, and the housing structure being constructed to allow access to the set of handcuff's locking mechanism;
at least two sensor modules having an interior portion of each sensor module located within the interior of the housing structure and an exterior portion of each sensor module protruding through the exterior of the housing structure, the exterior portion of each sensor module making contact with the wearer's wrist while the set of handcuffs are engaged in the locked position on the wearer, and each sensor module comprising a sensor adapted to measure at least one vitals measurement and continuously monitor that vitals measurement, wherein each sensor is selected from the group consisting of a SpO₂ sensor, photoplethysmography sensor, a bioimpedance sensor, a dermal temperature sensor, an electrocardiogram sensor, and an electrodermal activity sensor, and wherein at least two different types of sensors from the group are selected to be used;
a controller unit that is located within the interior of the housing structure, the controller unit being configured to relay instructional programs to the modules and units which cause the modules and units to operate, the controller unit receiving a vitals measurement from each sensor module and, upon detection of at least one vitals measurement outside of given parameters, producing at least one corresponding output signal related to the wearer's at least one vitals measurement outside of given parameters;
a communication unit that is located within the interior of the housing structure and receives the at least one corresponding output signal from the controller unit and is adapted to relay the at least one corresponding output signal to the law enforcement official to alert the law enforcement official of the wearer's medical issue via a speaker; and
a rechargeable power unit located within the interior of the housing structure that is accessed by a port on the exterior of the housing structure, provides power to the vitals monitoring apparatus's other modules and units, and having a receiver which connects with an exterior power source via the port.

2. The vitals monitoring apparatus of claim 1, wherein, upon activation of the vitals monitoring apparatus, the controller unit creates a profile, assigns an identifier to the profile, records data including the received sensor measured vitals measurements to the profile along with timestamps for the vitals measurements, and records any medical issue that occurs during the wearer's time wearing the activated vitals monitoring apparatus.

3. The vitals monitoring apparatus of claim 1, wherein, upon activation of the vitals monitoring apparatus, the controller unit creates a profile, assigns an identifier to the profile that is unique to the wearer, records data including the received sensor measured vitals measurements to the profile along with timestamps for the vitals measurements, and records any medical issue that occurs during the wearer's time wearing the activated vitals monitoring apparatus.

4. The vitals monitoring apparatus of claim 3, wherein the profile and data are assigned to a unique identifier to a specific wearer which may be used to link the wearer to the profile if a different vitals monitoring apparatus coupled with a set of handcuffs is placed on the wearer.

5. The vitals monitoring apparatus of claim 4, wherein the use of the profile assigned to a specific wearer allows for seamless transition of vitals monitoring between more than one vitals monitoring apparatus coupled with their own sets of handcuffs.

6. The vitals monitoring apparatus of claim 5, wherein the communication unit transmits the at least one vitals measurement and medical issue to a centralized system.

7. The vitals monitoring apparatus of claim 1, wherein the housing structure of the vitals monitoring apparatus is permanently affixed to the set of handcuffs.

8. A system including a vitals monitoring apparatus coupled to a set of handcuffs and a specialized external device, the system comprising:
a vitals monitoring apparatus coupled with a set of handcuffs comprising:
a housing structure having an exterior and interior, adapted to be coupled and uncoupled with the set of handcuffs used by a law enforcement official for detainment of a wearer, the interior of the housing structure containing a plurality of modules and units, the exterior of the housing structure constructed to generally have a complimentary shape to the set of handcuffs, the exterior of the housing structure configured to contact the wearer's wrist while the set of handcuffs is engaged in a locked position on the wearer's wrist during detainment, and the housing structure being constructed to allow access to the set of handcuff's locking mechanism;
at least two sensor modules having an interior portion of each sensor module located within the interior of the housing structure and an exterior portion of each sensor module protruding through the exterior of the housing structure, the exterior portion of each sensor module making contact with the wearer's wrist while the set of handcuffs are engaged in the locked position on the wearer, and each sensor module comprising a sensor adapted to measure at least one vitals measurement and continuously monitor that vitals measurement, wherein each sensor is selected from the group consisting of a SpO2 sensor, photoplethysmography sensor, a bioimpedance sensor, a dermal temperature sensor, an electrocardiogram sensor, and an electrodermal activity sensor, and wherein at least two different types of sensors from the group are selected to be used;
a controller unit that is located within the interior of the housing structure, the controller unit being configured to relay instructional programs to the modules and units which cause the modules and units to operate, the controller unit receiving a vitals measurement from each sensor module and, upon detection of at least one vitals measurement outside of given parameters, producing at least one corresponding output signal related to the wearer's at least one vitals measurement outside of given parameters;
a communication unit that is located within the interior of the housing structure and receives the at least one corresponding output signal from the controller unit and is adapted to relay the at least one corresponding output signal to the law enforcement official to alert the law enforcement official of the wearer's medical issue via a speaker; and
a rechargeable power unit located within the interior of the housing structure that is accessed by a port on the exterior of the housing structure, provides power to the vitals monitoring apparatus's other modules and units, and having a receiver which connects with an exterior power source via the port;
a specialized external device comprising:
a device attachment structure for attaching to at least one of the law enforcement official's belt, uniform, gear, and vehicle;
vitals monitoring apparatus connection method;
a speaker;
an apparatus holster structure for holstering with the vitals monitoring apparatus;
an apparatus charger for charging the vitals monitoring apparatus while holstered;
associated processors, memory, circuitry, and a power unit;
wherein the communication unit of the vitals monitoring apparatus transmits the wearer's at least one vitals measurement to the specialized external device;
wherein, upon detection of a vitals measurement outside of given parameters, the specialized external device sends an alert to the law enforcement official regarding the wearer's medical issue; and
wherein the vitals monitoring apparatus is adapted to charge while the vitals monitoring apparatus is holstered in apparatus holster structure of the specialized external device and the vitals monitoring apparatus is engaged with the apparatus charger.

9. The system of claim 8, wherein the specialized external device transmits the at least one vitals measurement and medical issue to a centralized system.

10. The system of claim 8, wherein the specialized external device is one or more devices selected from the group consisting of a body camera, a computer, a terminal, a small wearable device, a wearable accessory, a communication device, and combinations thereof.

11. The system of claim 8, further comprising the specialized external device having a display for providing the law enforcement official with at least one of: the wearer's vitals measurements, an alert about the wearer's medical emergency, an alert about the wearer's emerging medical issue, and an error code.

12. The system of claim 8, further comprising the specialized external device having an input method.

13. The vitals monitoring apparatus of claim 8, wherein, upon activation of the vitals monitoring apparatus, the vitals monitoring apparatus sends data from the sensors to the specialized external device, the specialized external device creates a profile, assigns an identifier to the profile, records data including the received sensor measured vitals measurements to the profile along with timestamps for the vitals measurements, and records any medical issue that occurs during the wearer's time wearing the activated vitals monitoring apparatus.

14. The vitals monitoring apparatus of claim 8, wherein, upon activation of the vitals monitoring apparatus, the vitals monitoring apparatus sends data from the sensors to the specialized external device, the specialized external device creates a profile, assigns an identifier to the profile that is unique to the wearer, records data including the received sensor measured vitals measurements to the profile along with timestamps for the vitals measurements, and records any medical issue that occurs during the wearer's time wearing the activated vitals monitoring apparatus.

15. The vitals monitoring apparatus of claim 14, wherein the profile and data are assigned to a unique identifier to a specific wearer which may be used to link the wearer to the profile if a different vitals monitoring apparatus coupled with a set of handcuffs is placed on the wearer.

16. The vitals monitoring apparatus of claim 15, wherein the use of the profile assigned to a specific wearer facilitates the seamless transition of vitals monitoring between more than one vitals monitoring apparatus.

17. The vitals monitoring apparatus of claim 8, wherein the specialized external device transmits the at least one vitals measurement and medical issue to a centralized system.

18. The vitals monitoring apparatus of claim 17, wherein the housing structure of the vitals monitoring apparatus is permanently affixed to the set of handcuffs.

19. A system including a vitals monitoring apparatus coupled with a set of handcuffs and a companion application, the system comprising:
the vitals monitoring apparatus coupled with a set of handcuffs comprising:
a housing structure having an exterior and interior, adapted to be coupled and uncoupled with the set of handcuffs used by a law enforcement official for detainment of a wearer, the interior of the housing structure containing a plurality of modules and units, the exterior of the housing structure constructed to generally have a complimentary shape to the set of handcuffs, the exterior of the housing structure configured to contact the wearer's wrist while the set of handcuffs is engaged in a locked position on the wearer's wrist during detainment, and the housing structure being constructed to allow access to the set of handcuff's locking mechanism;
at least two sensor modules having an interior portion of each sensor module located within the interior of the housing structure and an exterior portion of each sensor module protruding through the exterior of the housing structure, the exterior portion of each sensor module making contact with the wearer's wrist while the set of handcuffs are engaged in the locked position on the wearer, and each sensor module comprising a sensor adapted to measure at least one vitals measurement and continuously monitor that vitals measurement, wherein each sensor is selected from the group consisting of a SpO2 sensor, photoplethysmography sensor, a bioimpedance sensor, a dermal temperature sensor, an electrocardiogram sensor, and an electrodermal activity sensor, and wherein at least two different types of sensors from the group are selected to be used;
a controller unit that is located within the interior of the housing structure, the controller unit being configured to relay instructional programs to the modules and units which cause the modules and units to operate, the controller unit receiving a vitals measurement from each sensor module and, upon detection of at least one vitals measurement outside of given parameters, producing at least one corresponding output signal related to the wearer's at least one vitals measurement outside of given parameters;
a communication unit that is located within the interior of the housing structure and receives the at least one corresponding output signal from the controller unit and is adapted to relay the at least one corresponding output signal to the law enforcement official to alert the law enforcement official of the wearer's medical issue via a speaker; and
a rechargeable power unit located within the interior of the housing structure that is accessed by a port on the exterior of the housing structure, provides power to the vitals monitoring apparatus's other modules and units, and having a receiver which connects with an exterior power source via the port;
the companion application installed on at least one of the law enforcement official's personal device and a piece of law enforcement equipment;
wherein the companion application allows the law enforcement official to perform at least one of the following actions via a user interface: review the wearer's current vitals measurements, review trending vitals measurements, review maximum recorded vitals measurements, display the status of the vitals monitoring apparatus, review a log file created once the vitals monitoring apparatus was activated and the handcuffs engaged in the locked position on the wearer for detainment, add notes to the log file, and transmit data to a centralized server.

20. The system of claim 19 further comprising a specialized external device comprising:
a device attachment structure for attaching to one of the law enforcement official's belt, uniform, gear, and vehicle;
vitals monitoring apparatus connection method;
a speaker;
an apparatus holster structure for holstering with the vitals monitoring apparatus;
an apparatus charger for charging the holstered vitals monitoring apparatus;
associated processors, memory, circuitry, and a power unit;
wherein the communication unit of the vitals monitoring apparatus transmits the wearer's at least one vitals measurement to the specialized external device;
wherein, upon detection of a vitals measurement outside of given parameters, the specialized external device sends an alert to the law enforcement official regarding the wearer's medical issue; and
wherein the vitals monitoring apparatus is adapted to charge while the vitals monitoring apparatus is holstered in the specialized external device and engaged with the apparatus charger and wherein the companion application receives data from one or more of the specialized external device and the vitals monitoring handcuff.
